(19) United States
(12) Patent Application Publication
(10) Pub. No.: US 2024/0298972 A1
(43) Pub. Date: Sep. 12, 2024

(54) SYSTEMS AND METHODS FOR DETECTING HEART SOUNDS TO CONFIRM CARDIAC EVENTS

(71) Applicant: Pacesetter, Inc., Sylmar, CA (US)

(72) Inventors: Jong Gill, Valencia, CA (US); Gene A. Bornzin, Simi Valley, CA (US); Stuart Rosenberg, Woodbury, MN (US); Fujian Qu, San Jose, CA (US)

(21) Appl. No.: 18/665,687

(22) Filed: May 16, 2024

Related U.S. Application Data
(62) Division of application No. 16/262,625, filed on Jan. 30, 2019, now abandoned.

(51) Int. Cl. A61B 5/00; A61B 5/352; A61B 7/02

(52) U.S. Cl. CPC A61B 5/7221; A61B 5/352; A61B 5/6846; A61B 7/023; A61B 2562/0204

(57) ABSTRACT

The present disclosure provides systems and methods for confirming cardiac events based on heart sounds. An implantable medical device includes a sensing component configured to acquire a signal, and a processing component communicatively coupled to the sensing component, the processing component configured to receive the signal from the sensing component, analyze the received signal to detect the presence or absence of at least one heart sound, and confirm whether an initial detection of a cardiac event is accurate based on the detected presence or absence of the at least one heart sound.

### CROSS-REFERENCE TO RELATED APPLICATION

This application is a Divisional of application Ser. No. 16/262,625, filed Jan. 30, 2019, titled “SYSTEMS AND METHODS FOR DETECTING HEART SOUNDS TO CONFIRM CARDIAC EVENTS”, published as U.S. Published Application No. 2020/0237313, the subject matter of which is incorporated herein by reference in its entirety.

### FIELD OF THE DISCLOSURE

The present disclosure relates generally to implantable medical devices, and more specifically, to confirming cardiac events by detecting heart sounds using implantable medical devices.

### BACKGROUND ART

Implantable medical devices are commonly used to acquire and store biological data signals, such as cardiac signals and neurological signals. For example, the biological data signals may include an electrogram (EGM).

EGMs may be recorded and analyzed by an implantable medical device to detect one or more cardiac events. For example, EGMs may be analyzed to detect R waves or T waves in a cardiac cycle. Further, EGMs may be analyzed to diagnose pause (i.e., sinus pause) in a patient. However, at least some cardiac event detections from EGMs may be false positives (i.e., a detection of an event occurs but the actual event did not occur). Accordingly, it would be desirable to provide a system that is capable of confirming previously detected cardiac events.

### BRIEF SUMMARY OF THE DISCLOSURE

In one embodiment, the present disclosure is directed to an implantable medical device. The implantable medical device includes a sensing component configured to acquire a signal, and a processing component communicatively coupled to the sensing component, the processing component configured to receive the signal from the sensing component, analyze the received signal to detect the presence or absence of at least one heart sound, and confirm whether an initial detection of a cardiac event is accurate based on the detected presence or absence of the at least one heart sound.

In another embodiment, the present disclosure is directed to a method for confirming cardiac events using an implantable medical device. The method includes acquiring, using a sensing component of the implantable medical device, a signal, analyzing the signal, using a processing component of the implantable medical device, to detect the presence or absence of at least one heart sound, and confirming, using the processing component, whether an initial detection of a cardiac event is accurate based on the detected presence or absence of the at least one heart sound.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a block diagram of one embodiment of an implantable medical device (IMD).

FIG. 2 is a block diagram of one embodiment of a computing device that may be used with the IMD shown in FIG. 1.

FIG. 3 is a diagram illustrating heart sounds that occur during a typical cardiac cycle.

FIG. 4 is a diagram showing an example electrogram and an example signal acquired over the same period of time.

FIGS. 5A-5D illustrate a signal that is averaged over five beats, ten beats, twenty five beats, and thirty beats, respectively.

FIG. 6 is a graph illustrating signal amplitudes for multiple accurate R wave detections and multiple false detections.

FIG. 7A shows a signal acquired by an implantable medical device that is positioned on a stationary surface, exterior to a patient.

FIG. 7B shows a signal acquired by an implantable medical device that is implanted in a patient.

FIG. 8 is a graph illustrating sums of signals for first data in a baseline environment and for second data in an environment including heart sounds.

FIG. 9 shows a functional block diagram of an embodiment of the invention.

FIG. 10 is a flowchart which describes the processing performed by the electronic circuit on a generic pulse amplitude signal to provide arrhythmia discrimination.

FIGS. 11A and 11B are a flowchart which describes the processing performed by the electronic circuit on a generic pulse amplitude signal to provide optimization of sensing gain and threshold.

Corresponding reference characters indicate corresponding parts throughout the several views of the drawings.

### DETAILED DESCRIPTION OF THE DISCLOSURE

The present disclosure provides systems and methods for confirming cardiac events based on heart sounds. An implantable medical device includes a sensing component configured to acquire a signal, and a processing component communicatively coupled to the sensing component, the processing component configured to receive the signal from the sensing component, analyze the received signal to detect the presence or absence of at least one heart sound, and confirm whether an initial detection of a cardiac event is accurate based on the detected presence or absence of the at least one heart sound.

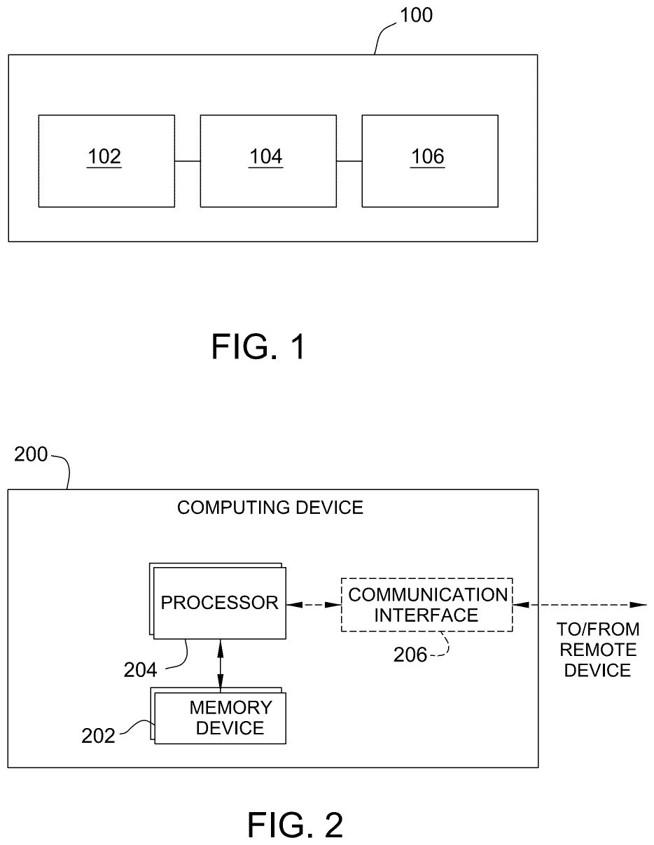

FIG. 1 is a block diagram of one embodiment of an implantable medical device (IMD) 100. IMD 100 is implantable in a patient and operable to acquire signals, process the signals to detect heart sounds, and generate an output based on the detected heart sounds. For example, in one embodiment, IMD 100 is one or more implantable cardiac monitors (ICM) operable to acquire signals, process the signals to detect heart sounds, and generate an output based on the detected heart sounds. In another embodiment, IMD 100 may be one or more implantable pacemakers operable to acquire signals, process the signals to detect heart sounds, and generate an output based on the detected heart sounds. Alternatively, IMD 100 may be any suitable implantable medical device capable of capturing any suitable biological data signals.

As shown in FIG. 1, IMD 100 includes a sensing component 102, a processing component 104, and an output component 106. Processing component 104 is communicatively coupled to sensing component 102 and output component 106. Sensing component 102 acquires raw (i.e., unfiltered) signals (including heart sounds) from the patient. Sensing component 102 includes any device capable of capturing or measuring signals that may be indicative of heart sounds.

For example, in some embodiments, sensing component 102 may be an accelerometer that measures vibration signals based on vibrations experienced by the accelerometer (e.g., mechanical vibrations generated by valve movement). In another example, in some embodiments, sensing component 102 may be an acoustic detector that measures acoustic signals indicative of heart sounds. For example, sensing component 102 may be an acoustic transducer as described in U.S. Pat. No. 6,477,406 entitled “EXTRAVASCULAR HEMODYNAMIC ACOUSTIC SENSOR”, which is incorporated by reference herein in its entirety. Further, sensing component 102 may be located in any suitable position. For example, in different embodiments, sensing component 102 may be extravascular, may be coupled to an implantable pulse generator housing, or may be incorporated in one or more leads of IMD 100.

Processing component 104 analyzes the signals acquired by sensing component 102, as described in detail herein. Specifically, processing component 104 analyzes the signals to detect heart sounds and confirm one or more cardiac events, as described in detail herein. Output component 106 then generates a suitable output based on the analysis performed by processing component 104.

Processing component 104 and/or output component 106 may be implemented using a computing device. For example, FIG. 2 illustrates one embodiment of a computing device 200 that may be used with IMD 100.

In this embodiment, computing device 200 includes at least one memory device 202 and a processor 204 that is coupled to memory device 202 for executing instructions. In some embodiments, executable instructions are stored in memory device 202. In the illustrated embodiment, computing device 200 performs one or more operations described herein by programming processor 204. For example, processor 204 may be programmed by encoding an operation as one or more executable instructions and by providing the executable instructions in memory device 202.

Processor 204 may include one or more processing units (e.g., in a multi-core configuration). Further, processor 204 may be implemented using one or more heterogeneous processor systems in which a main processor is present with secondary processors on a single chip. In another illustrative example, processor 204 may be a symmetric multi-processor system containing multiple processors of the same type. Further, processor 204 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In the illustrated embodiment, memory device 202 is one or more devices that enable information such as executable instructions and/or other data to be stored and retrieved. Memory device 202 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 202 may be configured to store, without limitation, application source code, application object code, source code portions of interest, object code portions of interest, configuration data, execution events and/or any other type of data.

Computing device 200, in the illustrated embodiment, includes a communication interface 206 coupled to processor 204. Communication interface 206 communicates with one or more remote devices, such as a clinician or patient programmer. To communicate with remote devices, communication interface 206 may include, for example, a radio-frequency (RF) adapter, such as a Bluetooth or medical implant communication system (MICS) adapter, and/or a near-field telecommunications adapter, also referred to as inductive telemetry.

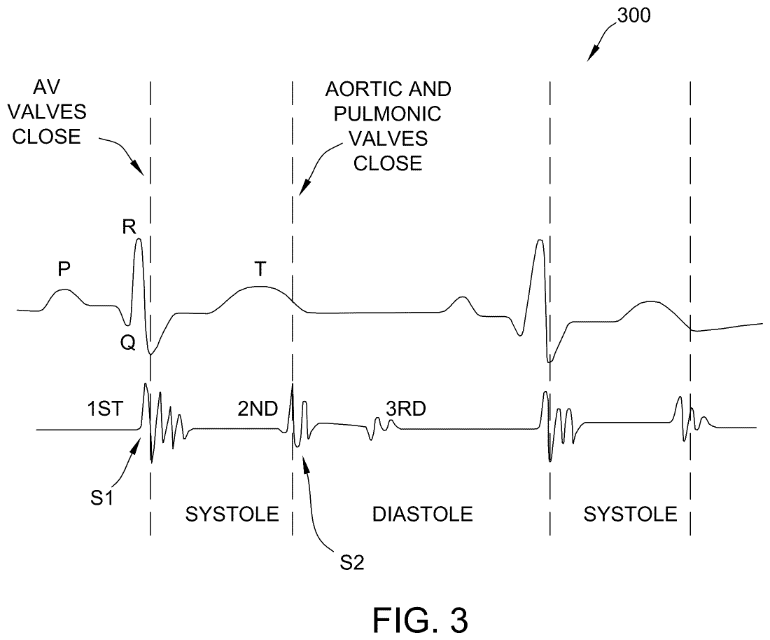

FIG. 3 is a diagram 300 illustrating heart sounds that occur during a typical cardiac cycle. As shown in FIG. 3, in a typical cardiac cycle, the atrio-ventricular (AV) valves, such as the mitral and tricuspid valves of the heart, close around the end of an R wave. The closing of the AV valves generates a first heart sound (S1) and marks the start of systole. Further, in a typical cardiac cycle, around the end of a T wave, the aortic and pulmonic valves of the heart close. The closing of the aortic and pulmonic valves generates a second heart sound (S2) and marks the end of systole. S1 and S2 are detectable in signals acquired using sensing component 102 (shown in FIG. 1), as described in detail herein. Further the detection of at least one of S1 and S2 (or the absence of the detection of at least one of S1 and S2) can be used to confirm different cardiac events, as described herein.

For example, the detection of S1 may be used to confirm the presence of an R wave during a cardiac cycle. Specifically, IMD 100 uses the detection of S1 to confirm an initial detection of an R wave. As will be appreciated by those of skill in the art, the initial detection of the R wave may be made, for example, from analyzing an electrogram (EGM). The EGM may be acquired and analyzed, for example, by IMD 100. In such embodiments, IMD 100 includes any additional sensors and/or circuitry necessary to acquire and analyze the EGM. Alternatively, the EGM may be acquired and analyzed by a separate device to perform the initial detection of the R wave.

Once an R wave is initially detected, IMD 100 can confirm (or deny) whether an R wave actually occurred based on detecting S1. That is, due to noise and/or other complications, the initial R wave detection may be false (i.e., the “detected” R wave may not have actually occurred). For example, in some situations, a sensing threshold for detecting an R wave in an EGM may be lowered (e.g., due to pocket instability of an implanted device). However, lowering the sensing threshold may result in noise in the EGM improperly being detected as an R wave. Accordingly, IMD 100 uses detection of S1 to determine whether or not the initial R wave detection is accurate.

In one embodiment, IMD 100 determines whether or not an initial R wave detection is accurate based on whether or not S1 is detected. Specifically, suppose an R wave is detected as occurring at a first time. To confirm the detection, processing component 104 analyzes signals acquired by sensing component 102 during a predetermined period of time that runs from the first time. For example, processing component 104 may monitor signals acquired within 100 milliseconds (ms) of the first time.

If processing component 104 analyzes the signals and detects S1 within the predetermined period of time, processing component 104 confirms the initial R wave detection. However, if processing component 104 does not detect S1 within the predetermined period of time, processing component 104 determines that the initial R wave detection was false.

In one embodiment, when the initial R wave detection is determined to be false, processing component 104 causes output component 106 to generate a corresponding alert. The generated alert may be any alert that facilitates alerting a user of the false R wave detection. For example, the alert may generated and transmitted (e.g., via communication interface 206) to a remote device, such as a clinician or patient programmer. The transmitted alert may cause the remote device to emit a sound or display a message that notifies the user of the false R wave detection. In some embodiments, any alerts generated may also be stored on the IMD 100 for future analysis.

Further, in some embodiments, the sensing threshold for detecting an R wave in an EGM may be adjusted based on the presence of S1. For example, if the sensing threshold is detecting a relatively high number of R waves that are false positives (i.e., that are not confirmed by an S1 detection), the sensing threshold may be raised to be more sensitive.

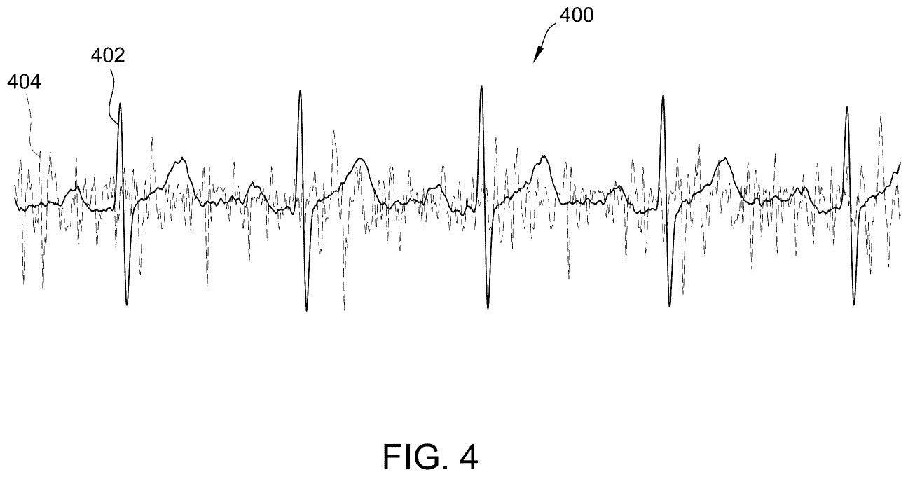

In some situations, the signals acquired by sensing component 102 may have relatively high noise levels, making it difficult for processing component 104 to detect S1 from only a single cardiac cycle. For example, FIG. 4 is a diagram 400 showing an example ECG 402 and an example signal 404 (e.g., a vibration signal) acquired over the same period of time. As shown in FIGS. 4, S1 and S2 are present in signal 404, but signal 404 is fairly noisy.

Accordingly, in some embodiments, processing component 104 performs ensemble averaging over a number of cardiac cycles to accurately detect S1 and S2. That is, processing component 104 averages a signal over a number of cardiac signals and analyzes the averaged signal to detect S1 and S2. The initially detected R wave can be used by processing component 104 as a reference point when averaging the signal over multiple cardiac cycles. That is, each first time (i.e., when an R wave is initially detected) can be used by processing component 104 to demarcate the beginning of a discrete cardiac cycle.

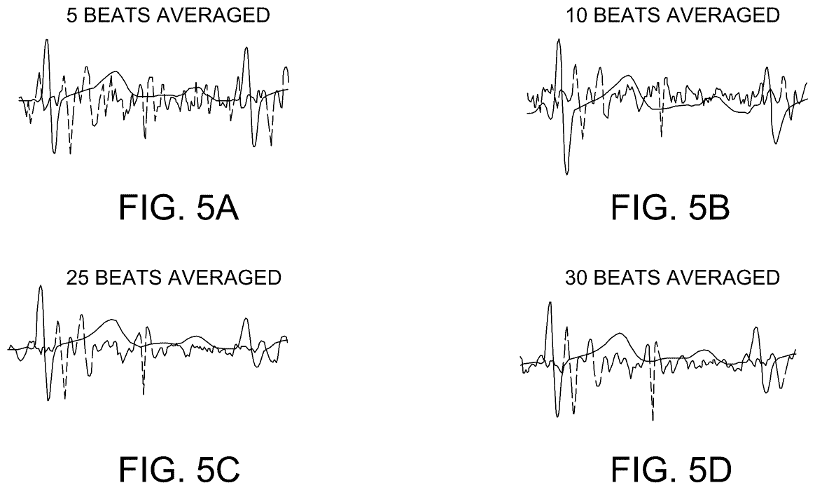

For example, FIGS. 5A-5D illustrate a signal (e.g., a vibration signal) acquired by sensing component 102 that is averaged over five beats, ten beats, twenty five beats, and thirty beats, respectively. As demonstrated by FIGS. 5A-5D the more cycles over which the signal is averaged, the clearer S1 and S2 are in the signal. However, the more cycles used for averaging, the longer it necessarily takes to determine whether S1 and/or S2 are present. Accordingly, in such embodiments, the number of cycles averaged should be balanced against the desired detection time. In some embodiments, the number of cycles averaged by processing component 104 is programmable and can be modified by a user (e.g., using a clinician programmer in communication with IMD 100). Additionally, in situations where R wave detections are not accurate the intervals between any alleged R waves and S1 will vary, leading to reduced S1 and/or S2 amplitudes when ensemble averaging is performed. Thus, the S1 and/or S2 amplitudes from ensemble-averaged signals are a reliable marker for appropriate R wave detections.

In one embodiment, to determine whether or not S1 is detected in the signal, processing component 104 compares the amplitude of the signal (e.g., the averaged signal) for the predetermined period of time to a threshold value. If the amplitude exceeds the threshold value, S1 is detected. If the amplitude does not exceed the threshold value, S1 is not detected. The threshold value may be programmable such that it is modifiable by a user (e.g., using a clinician programmer in communication with IMD 100) to adjust the sensitivity of S1 detections. Further, in some embodiments, the threshold value may be dynamic. That is the threshold value may be updated over time based on detected amplitudes of S1 (and, in some embodiments, S2).

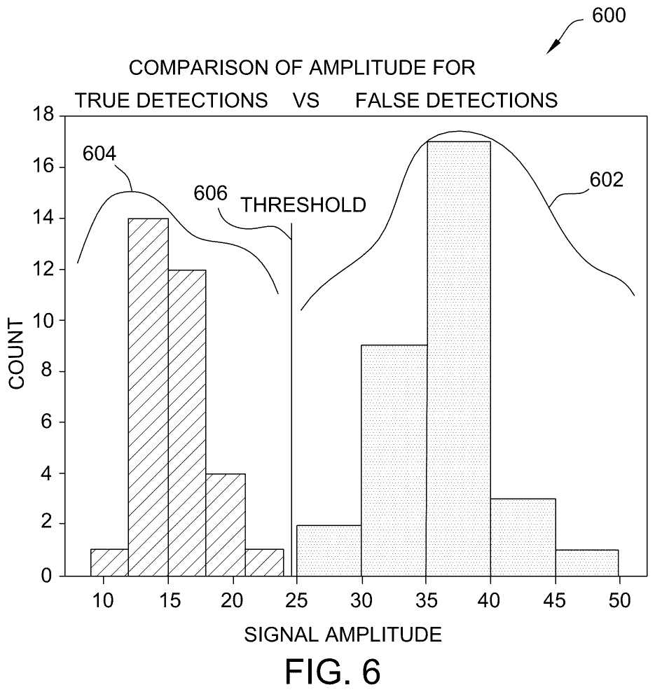

FIG. 6 is a graph 600 illustrating signal amplitudes (e.g., vibration signal amplitudes) for multiple accurate R wave detections 602 and multiple false R wave detections 604. As shown in FIG. 6, the signal amplitude for accurate R wave detections 602 is substantially higher than the signal amplitude for false R wave detections 604. Accordingly, by selecting an appropriate threshold value (e.g., threshold 606), processing component 104 can accurately identify when S1 is present and when S1 is not present (and accordingly, accurately confirm or deny R wave detections). Further, in some embodiments, the threshold value may be automatically and dynamically determined by processing component 104. For example, processing component 104 may monitor and record signal amplitudes over time to determine an appropriate threshold value. Further, if sensing component 102 is moved, processing component may determine an updated threshold value.

The systems and methods described herein can also be used to confirm or deny initial T wave detections (in addition to or instead of R wave detections). As explained above, S2 occurs at the end of a T wave. Accordingly, using the same or substantially similar techniques described above in association with S1, processing component 104 may confirm or deny initial T wave detections based on whether or not S2 is detected. As these techniques are largely identical to those described in association with S1, those of skill in the art will appreciate these techniques without further description herein.

Further, the systems and methods described herein may be used to confirm an initial pause detection. Pause (also referred to as sinus pause) is indicated by an absence of an R wave. Accordingly, IMD 100 or another device may initially detect a pause, for example, by analyzing an EGM. If an actual pause has occurred, S1 and S2 will be absent. In contrast, if an actual pause does not occur, S1 and S2 should be present. Accordingly, by analyzing signals for S1 and S2, an initial pause detection can be confirmed or denied based on whether S1 and S2 are detected. Specifically, in one embodiment, if processing component 104 detects S1 and S2, the initial pause detection is rejected as false. In contrast, if processing component 104 does not detect S1 and S2, the initial pause detection is confirmed.

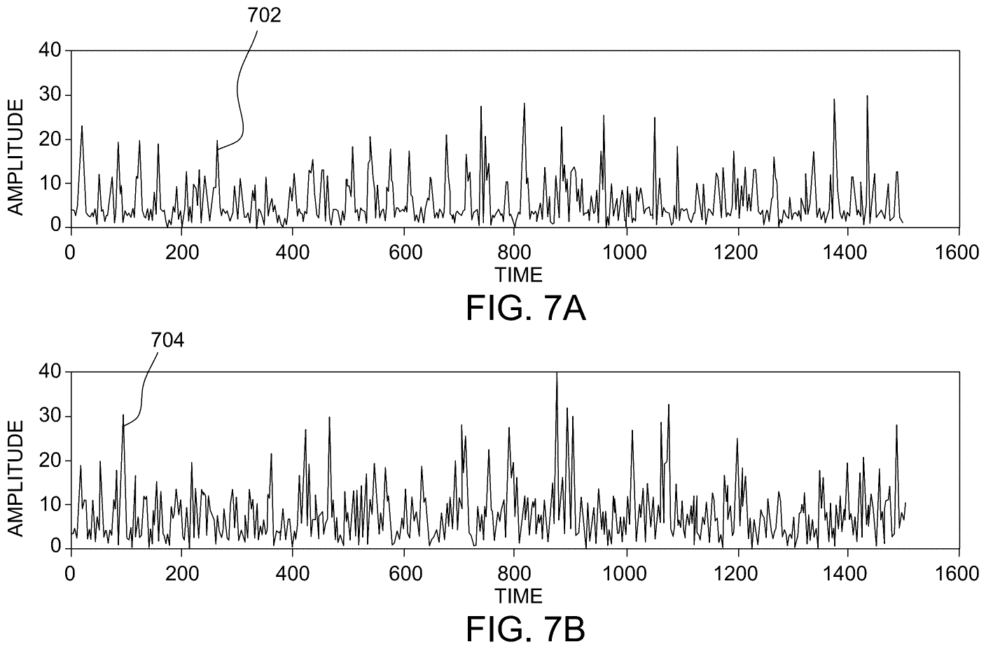

As discussed above, signals acquired using sensing component 102 may be relatively noisy. For example, FIG. 7A shows an signal 702 (e.g., a vibration signal) acquired by sensing component 102 when IMD 100 is positioned on a stationary surface, exterior to the patient. FIG. 7B, in contrast, shows a signal 704 (e.g., a vibration signal) acquired by sensing component 102 when IMD 100 is implanted in a patient. As demonstrated by FIG. 7A, sensing component 102 is relatively sensitive, picking up significant noise even when positioned on a stationary surface, exterior to the patient. Accordingly, signal 704 includes heart sound features detected on top of the ambient noise.

However, unlike R wave detection confirmation (described above), when confirming an initial pause detection, processing component 104 cannot use ensemble averaging to reduce noise. Specifically, as described above, for R wave detection confirmation, processing component 104 may use the first times (at which R waves are detected) as a trigger to identify cardiac cycles. In contrast, the initial pause detection is based on the absence of an R wave. Accordingly, R wave detections cannot be used a reference by processing component 104 for ensemble averaging when confirming pause detections.

Accordingly, in one embodiment, processing component 104 detects S1 and S2 (or the absence of S1 and S2) by determining a sum of a signal acquired by sensing component 102 over a predetermined duration. Because heart sound features are present if the pause detection is false, the sum of the signal should be larger when the pause detection is false as opposed to when the pause detection is accurate.

Specifically, in one embodiment, once a pause is initially detected, processing component 104 sums the signal acquired by sensing component 102 over a predetermined duration. Prior to summing, in some embodiments, the signal is rectified so that the sum accounts for both positive and negative values. Processing component 104 then compares the sum to a threshold value. If the sum exceeds the threshold value, processing component 104 determines that S1 and S2 are present, and accordingly, determines the initial pause detection is false. In contrast, if the sum does not exceed the threshold value, processing component 104 determines that S1 and S2 are not present, and confirms the pause detection. The length of the duration over which the sum is calculated and the threshold value may programmable (e.g., using a clinician programmer in communication with IMD 100) to adjust sensitivity. For example, the duration may be approximately 3 seconds. Further, the threshold value may be dynamic, such that it is updated overtime based on confirmed S1 and S2 detections. In one example, the threshold value is based on a distribution of historical values of the summed signal. For example, the threshold value may be the average signal value minus 3*σ, where σ is the standard deviation of obtained signal values.

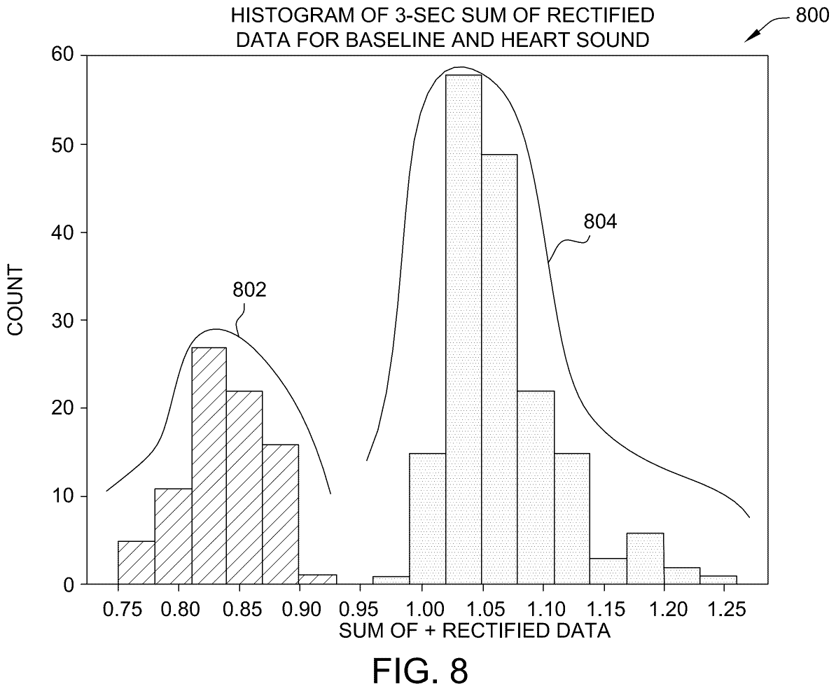

FIG. 8 is a graph 800 illustrating sums of signals (e.g., vibration signals) for first data 802 in a baseline environment (i.e., an environment without S1 and S2) and for second data 804 in an environment including S1 and S2. As shown in FIG. 8, the sums for first data 802 are substantially lower than the sums for second data 804. Accordingly, by selecting an appropriate threshold value between first data 802 and second data 804, processing component 104 can accurately identify from the sum whether S1 and S2 are present (and accordingly, accurately confirm or deny pause detections).

In one embodiment, when the initial pause detection is confirmed, processing component 104 causes output component 106 to generate a corresponding alert. The generated alert may be any alert that facilitates alerting a user of the confirmed pause detection. For example, the alert may generated and transmitted (e.g., via communication interface 206) to a remote device, such as a clinician or patient programmer. The transmitted alert may cause the remote device to emit a sound or display a message that notifies the user of the confirmed pause detection. In some embodiments, any alerts generated may also be stored on the IMD 100 for future analysis.

Accordingly, the systems and method described herein facilitate confirming cardiac events by detecting heart sounds. An implantable medical device includes a sensing component configured to acquire a signal, and a processing component communicatively coupled to the sensing component, the processing component configured to receive the signal from the sensing component, analyze the received signal to detect the presence or absence of at least one heart sound, and confirm whether an initial detection of a cardiac event is accurate based on the detected presence or absence of the at least one heart sound.

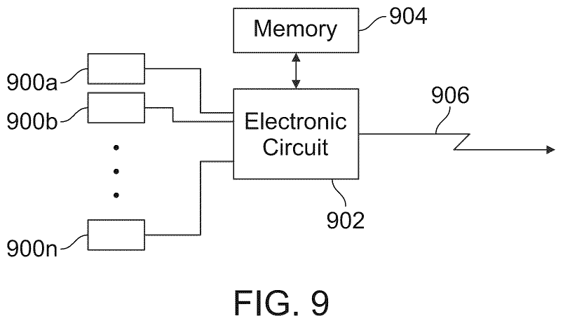

A functional block diagram of hemodynamic sensors incorporated into an implantable device is shown in FIG. 9. One or a plurality of sensors 900a, 900b, . . . 900n is connected to an electronic circuit 902. When the implantable device is a pacemaker or ICD, the electronic circuit 902 is further connected to a cardiac lead 906, capable of delivering pace stimuli or antitachycardia therapy to the heart. The diagrammatic representation of sensors 900a, . . . , 900n and lead 906 illustrates the functional distinction, but does not imply a separate physical embodiment. The device includes a memory 904 and electronic circuit 902 that contains a low-power microprocessor.

Conventional implantable cardioverter defibrillators (ICDs) perform discrimination through analysis of the intracardiac electrogram, where features such as rate, interval regularity, and QRS morphology are analyzed in order to identify the underlying rhythm. This approach is convenient because electrical sensing can be easily performed using the same leads that the device requires to deliver therapy.

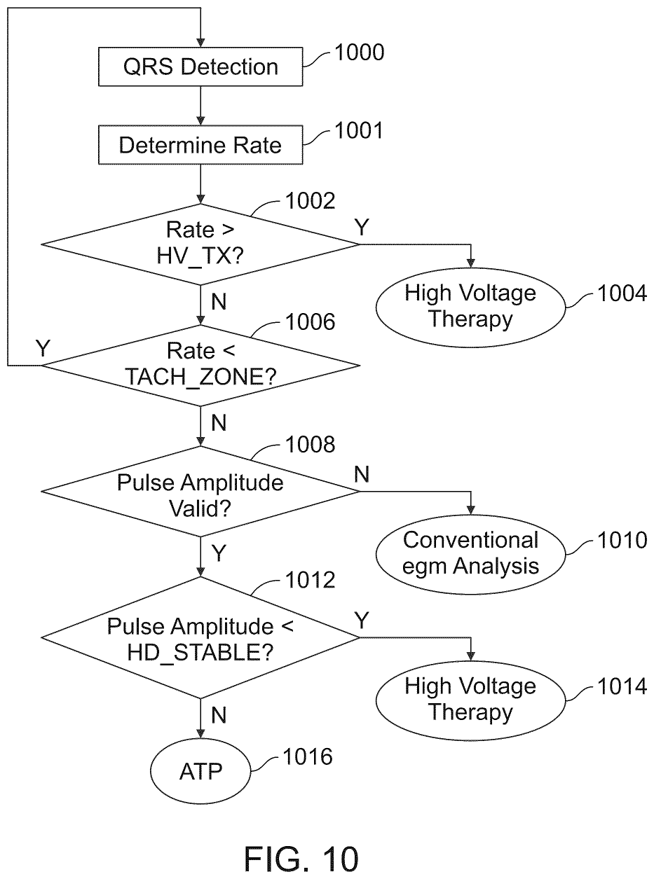

The algorithm which performs tachyarrhythmia discrimination using hemodynamic sensing is presented in FIG. 10. After the detection of a sensed event, such as a ventricular depolarization, at step 1000, the current heart rate is determined at step 1001. If the rate is above a predetermined threshold for high voltage therapy, HV_TX, tested at step 1002, then control is transferred to a conventional algorithm which immediately delivers high voltage therapy, step 1004. Otherwise, the rate is compared to TACH_ZONE, which defines the low end of the tachycardia rate zone, at step 1006. If the rate is below this threshold then control returns to step 1000, and the algorithm awaits the detection of the next sensed event.

Otherwise, the rate is in the overlap zone. The algorithm therefore delivers therapy based on the hemodynamic status of the patient. The validity of the pulse amplitude, determined in the preferred embodiment of the pulse amplitude algorithm, such as those described in conjunction with FIG. 6 of U.S. Pat. No. 6,477,406, incorporated by reference in its entirety herein, is first tested at step 1008. This test uses the error flag which is set in the preferred embodiment of the pulse amplitude algorithm when significant artifact is present in the underlying signal. If the pulse amplitude is invalid, then control passes to an algorithm that performs conventional electrogram analysis, step 1010. If the value is valid, then it is tested against a predetermined threshold for hemodynamic stability at step 1012. If the pulse amplitude is less than this value, then the high ventricular rate has compromised the hemodynamic stability of the patient to a degree that warrants high voltage therapy, regardless of the precise nature of the arrhythmia. This is delivered by passing control to a conventional high voltage therapy algorithm at step 1014. On the other hand, if the pulse amplitude exceeds the threshold, the patient is maintaining sufficient hemodynamic stability to attempt low voltage therapy. Control therefore passes to a conventional anti-tachycardia pacing algorithm at step 1016.

In still other alternate embodiments the analysis examines the pattern of pulse amplitudes to identify the nature of the arrhythmia. For example, the detection of pulse amplitudes that vary on a beat-to-beat basis suggests that conducted AF is present. Similarly, other analysis using the pulse amplitude signal can better identify the nature of the underlying rhythm. The pulse amplitude signal can of course be used alone or in conjunction with other hemodynamic or electrical signals, such as the intracardiac electrogram.

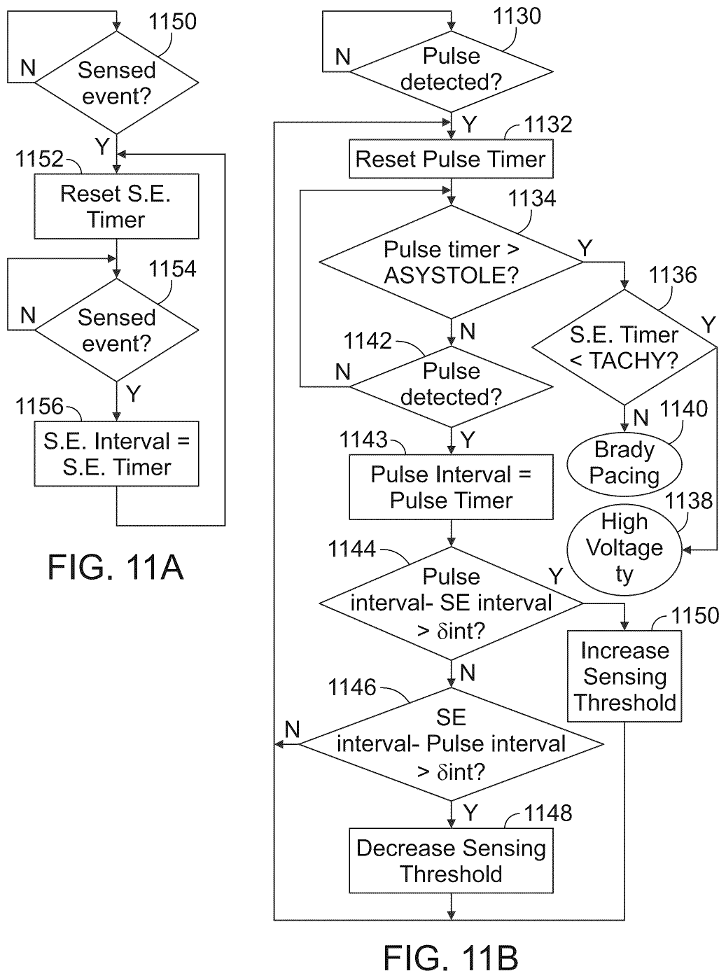

The threshold/gain optimization algorithm makes use of a record of intervals between systolic pulses and an independently obtained record of intervals between electrical sensed events. The algorithm by which the record of sensed events is obtained is illustrated in FIG. 11a. It is initialized with the first detection of an electrical sensed event at step 1150. This causes the free-running sensed event timer to be reset at step 1152. Upon detection of the next sensed event at step 1154, the contents of the sensed event timer are transferred to the sensed event interval register at step 1156, and control returns to step 1152. As seen in FIG. 11b, independent of the execution of the algorithm that maintains the sensed event interval register, the algorithm that performs sensing threshold optimization begins with the first detection of a systolic pulse or cardiac contraction at step 1130. The crossing of a threshold, predetermined to be much lower than the amplitude generated by a systolic contraction or pulse but much greater than baseline noise, is detected in analog circuitry using a comparator, which is recognized at step 1130 as the detected pulse. The pulse detection causes the free-running pulse timer to be reset at step 1132. At step 1134, the pulse timer is compared against a predetermined duration ASYSTOLE, set to 1.5 seconds in the preferred embodiment. If the pulse timer exceeds this value then the most recent sensed event interval is compared at step 1136 to a predetermined duration TACHY, set to 500 msec in the preferred embodiment. If the sensed event timer is less than TACHY then a hemodynamically unstable arrhythmia is diagnosed and control is transferred to the algorithm that delivers high voltage therapy at step 1138. On the other hand, if the sensed event interval is greater than TACHY then a hemodynamically unstable bradycardia is diagnosed and control is transferred to the algorithm that delivers brady pacing therapy at step 1140. Returning to step 1134, if the pulse timer is less than ASYSTOLE, then a hemodynamically stable condition is diagnosed, and control is passed to step 1142, where the detection of a pulse is tested. If no pulse is present then control returns to step 1134. If a pulse was detected then the contents of the pulse timer is transferred to a pulse interval register at step 1143. The difference between the just-obtained pulse interval and the most recent sensed event interval is compared to the predetermined value δint at step 1144. Testing immediately after a systolic pulse is advantageous because, as illustrated in FIG. 9b of U.S. Pat. No. 6,477,406, incorporated by reference in its entirety herein, the most recently obtained electrical and hemodynamic intervals are derived from the same cardiac cycle. Because intrinsic variability is present, the intervals are not required to be equal at step 1144, rather, the predetermined value δint, preferably set to 75 msec, is used to account for this variability. If the test at step 1144 is positive, so that the most recent hemodynamic interval exceeds the most recent electrical interval by more than δint, then oversensing is deemed to be present in the electrical signal, and the electrical sensing threshold is increased at step 1150, or alternatively, the electrical amplifier gain is decreased. If the test at step 1144 is negative, then the difference between the most recent sensed event interval and the most recent pulse interval is compared to δint. If the test is positive, then undersensing is deemed to be present, so the electrical sensing threshold is lowered at step 1148, or, equivalently, the gain is increased. Control returns to step 1132 after steps 1148, 1150, and a negative result at step 1146.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An implantable medical device comprising:
a heart sound (HS) sensing component configured to acquire a signal; and
a sensor and circuitry configured to acquire an electrogram (EGM);
memory configured to store executable instructions; and
a processor communicatively coupled to the sensing component, the processor, when executing the instructions, is configured to:
initially detect a cardiac event based on the EGM;
receive the signal from the sensing component;
analyze the received signal to detect the presence or absence of at least one heart sound;
confirm whether the initial detection of the cardiac event is accurate based on the detected presence or absence of the at least one heart sound; and
deliver therapy associated with the cardiac event based on the confirmation or denial of the initial detection of the cardiac event.

2. The implantable medical device of claim 1, wherein to analyze the received signal to detect the presence or absence of a second heart sound, the processor is configured to:
average the signal over a plurality of cardiac cycles;
analyze the averaged signal to detect the presence or absence of the second heart sound; and
in response to the detected presence of the second heart sound, confirm the initial detection of a T wave is accurate.

3. The implantable medical device of claim 1, wherein to analyze the received signal to detect the presence or absence of a second heart sound, the processor is configured to:
determine an amplitude of the received signal;
compare the amplitude to a threshold value;
determine that the second heart sound is present when the amplitude exceeds the threshold value; and
determine that the second heart sound is absent when the amplitude does not exceed the threshold value.

4. The implantable medical device of claim 3, wherein to confirm whether an initial detection of a T wave is accurate, the processor is configured to:
confirm the initial detection of the T wave is accurate when the second heart sound is present; and
confirm the initial detection of the T wave is inaccurate when the second heart sound is absent.

5. The implantable medical device of claim 1, wherein to analyze the received signal, the processor is configured to analyze the received signal to detect the presence or absence of a second heart sound, and wherein to confirm whether an initial detection of a cardiac event is accurate, the processor is configured to confirm whether an initial detection of a T wave is accurate.

6. The implantable medical device of claim 1, wherein to analyze the received signal, the processor is configured to analyze the received signal to detect the presence or absence of a first heart sound and a second heart sound, and wherein to confirm whether an initial detection of a cardiac event is accurate, the processor is configured to confirm whether an initial detection of a pause is accurate.

7. The implantable medical device of claim 1, wherein to detect the presence or absence of a first heart sound and a second heart sound, the processor is configured to:
sum the received signal over a predetermined duration;
compare the summed signal to a threshold value;
determine the first and second heart sound are present when the summed signal exceeds the threshold value; and
determine the first and second heart sounds are absent when the summed signal does not exceed the threshold value.

8. The implantable medical device of claim 7, wherein the threshold value is dynamic over time based on confirmed first and second heart sound detections.

9. The implantable medical device of claim 7, wherein to confirm whether an initial detection of a pause is accurate, the processor is configured to:
confirm the initial detection of the pause is accurate when the first and second heart sounds are absent; and
confirm the initial detection of the pause is inaccurate when the first and second heart sounds are present.

10. The implantable medical device of claim 1, wherein the signal is one of an acoustic signal and a vibration signal.

11. A method for confirming cardiac events using an implantable medical device, the method comprising:
acquiring, using a sensor and circuitry of the implantable medical device, an electrogram (EGM);
initially detecting a cardiac event based on the EGM;
acquiring, using a sensing component of the implantable medical device, a signal;
analyzing the signal, using a processor of the implantable medical device, to detect the presence or absence of at least one heart sound;
confirming, using the processor, whether the initial detection of the cardiac event is accurate based on the detected presence or absence of the at least one heart sound; and
delivering therapy associated with the cardiac even based on the confirmation or denial of the initial detection of the cardiac event.

12. The method of claim 11, wherein analyzing the acquired signal to detect the presence or absence of a second heart sound comprises:
averaging the signal over a plurality of cardiac cycles;
analyzing the averaged signal to detect the presence or absence of the second heart sound; and
in response to the detected presence of the second heart sound, confirming the initial detection of a T wave is accurate.

13. The method of claim 11, wherein analyzing the received signal to detect the presence or absence of a second heart sound comprises:
determining an amplitude of the received signal;
comparing the amplitude to a threshold value;
determining that the first second sound is present when the amplitude exceeds the threshold value; and
determining that the second heart sound is absent when the amplitude does not exceed the threshold value.

14. The method of claim 13, wherein confirming whether an initial detection of a T wave is accurate comprises:
confirming the initial detection of the T wave is accurate when the second heart sound is present; and
confirming the initial detection of the T wave is inaccurate when the second heart sound is absent.

15. The method of claim 11, wherein analyzing the acquired signal comprises analyzing the acquired signal to detect the presence or absence of a second heart sound, and wherein confirming whether an initial detection of a cardiac event is accurate comprises confirming whether an initial detection of a T wave is accurate.

16. The method of claim 11, wherein analyzing the acquired signal comprises analyzing the acquired signal to detect the presence or absence of a first heart sound and a second heart sound, and wherein confirming whether an initial detection of a cardiac event is accurate comprises confirming whether an initial detection of a pause is accurate.

17. The method of claim 11, wherein detecting the presence or absence of a first heart sound and a second heart sound comprises:
summing the acquired signal over a predetermined duration;
comparing the summed signal to a threshold value;
determining the first and second heart sound are present when the summed signal exceeds the threshold value; and
determining the first and second heart sounds are absent when the summed signal does not exceed the threshold value.

18. The method of claim 17, wherein the threshold value is dynamic over time based on confirmed first and second heart sound detections.

19. The method of claim 17, wherein confirming whether an initial detection of a pause is accurate comprises:
confirming the initial detection of the pause wave is accurate when the first and second heart sounds are absent; and
confirming the initial detection of the pause wave is inaccurate when the first and second heart sounds are present.

20. The method of claim 11, wherein the signal is one of an acoustic signal and a vibration signal.
